(19) United States
(12) Patent Application Publication
(10) Pub. No.: US 2011/0065985 A1
(43) Pub. Date: Mar. 17, 2011

(54) ENDOSCOPIC INSTRUMENT

(75) Inventor: Frank WEHRHEIM, Bretten (DE)

(73) Assignee: RICHARD WOLF GMBH, Knittlingen (DE)

(21) Appl. No.: 12/881,294

(22) Filed: Sep. 14, 2010

(30) Foreign Application Priority Data
Sep. 14, 2009 (DE) 10 2009 041 510.6

(51) Int. Cl. A61B 1/00

(52) U.S. Cl. 600/106

(57) ABSTRACT

An endoscopic instrument has a proximally arranged operating part, a distally arranged instrument head and a shaft connecting the operating part to the instrument head. A number of instrument channels guided through the shaft open out on the instrument head. These instrument channels run obliquely outwardly in the distal direction with respect to a central axis of the instrument head in a section of the channels that adjoins the distal openings thereof.

### BACKGROUND OF THE INVENTION

The invention relates to an endoscopic instrument having a proximally arranged operating part, a distally arranged instrument head, a shaft connecting the operating part to the instrument head, and a number of instrument channels guided through the shaft opening out on the instrument head.

Instruments of the type in question are used in the medical sector and serve for diagnosis and/or therapy purposes within the body interior. Such instruments have a shaft that is guided into the body interior via natural or artificially produced channels. It is usual for an instrument head to be provided at the distal end of the shaft, with observation and illumination arrangements being arranged on the distal end face of the instrument head. In general, at least one instrument channel for an auxiliary instrument guided through the shaft also opens out at the end face.

In surgical interventions performed with the evermore frequently applied NOTES (natural orifice transluminal endoscopic surgery) operation method in particular, it is necessary for a plurality of auxiliary instruments to be guided to the operating space via the shaft and the instrument head adjoining the latter, and for the auxiliary instruments to be made available there, in part at the same time. Accordingly, endoscopic instruments used for this purpose must have a plurality of instrument channels for auxiliary instruments, which instrument channels open out on the instrument head. Frequently, the aim is first to fan open the auxiliary instruments toward the outside, which auxiliary instruments emerge from the instrument on the end face of the instrument head and normal to the end face, in order subsequently, like the three-finger grippers used in handling machines, to bring them together again in the direct vicinity of the operation object. The fanning-out of the auxiliary instruments outside of the instrument requires a relatively large space, and so this fanning-out is only possible in correspondingly large operating spaces. Moreover, the need for a plurality of instrument channels is disadvantageous in respect of miniaturizing shaft and instrument head cross sections, which is otherwise aimed for in endoscopic instruments.

### BRIEF SUMMARY OF THE INVENTION

Against this background it is the object of the invention to develop an endoscopic instrument with a plurality of instrument channels, which has a comparatively small shaft and instrument head cross section and can be operated and controlled in relatively small operating spaces.

This object is achieved by an endoscopic instrument of type described at the outset, characterized in that the instrument channels run obliquely outwardly in the distal direction with respect to a central axis of the instrument head in a section of the channels that adjoins the distal openings thereof. Advantageous embodiments of this instrument are to be deduced from the following description and drawings. Herein, according to the invention, the features described can each, on their own or else in combination, further refine the solution according to the invention.

The endoscopic instrument according to the invention, which is preferably an instrument used in medicine, but can also be a so-called technoscope for use in cavities of machines and the like, as is conventional, has a proximally arranged operating part and a distally arranged instrument head. A shaft, which should be understood both as a rigid and a flexible hollow shaft within the scope of the invention, with a tube also being able to be a shaft, connects the operating part to the instrument head. A number of instrument channels are guided through the shaft. The instrument channels open out on the instrument head. The instrument channels are used to guide auxiliary instruments to an operating space situated outside of the distal end of the instrument head, when the endoscopic instrument is in use.

According to the invention, the instrument channels run obliquely outwardly in the distal direction with respect to a central axis of the instrument head in a section of the channels that adjoins the distal openings thereof. Herein, the sections of the instrument channels aligned obliquely with respect to the central axis of the instrument head are advantageously arranged completely within the instrument head. The openings of the instrument channels are preferably not formed in the region of the end face of the instrument head, but on an edge region adjoining the end face in the circumferential direction. This is advantageous to the extent that thus there is enough space available on the end face for arranging an observation arrangement and an illumination arrangement for the purpose of navigating the instrument according to the invention. Moreover, if need be, additional arrangements, such as force sensors for recording forces acting in the distal direction and rinsing and suction arrangements, can also be arranged on this end face. However, unlike in previously known instruments of this type, as not the instrument channels provided for guiding auxiliary instruments are opening out on the end face of the instrument head, then the cross section of the instrument head can typically have smaller dimensions than in the previously known instruments. A further advantage of the outwardly angled distal end sections of the instrument channels can be seen in the fact that a desired fanning-out of the auxiliary instruments can already be initiated within the instrument head, as described above. The auxiliary instruments can be brought together again in a correspondingly tighter manner around the distal end of the instrument head, and so significantly less space is required for fanning-out the auxiliary instruments compared to the previously used endoscopic instruments.

Typically, the auxiliary instruments used in the endoscopic instrument according to the invention are such instruments that have a shaft, which at least in one distal end section can be deflected obliquely with respect to the longitudinal extent of the shaft in at least one plane. These auxiliary instruments can be guided particularly easily in the instrument channels if the instrument channels in the instrument head, as provided in an advantageous embodiment of the invention, transition in an arched manner from a proximal-side section aligned parallel to the central axis of the instrument head to a distal-side section aligned obliquely with respect to the central axis of the instrument head; that is, if there are no abrupt directional changes, formed by angled sections or otherwise, in the instrument channels at which the auxiliary instruments could otherwise jam.

Preferably the instrument head tapers off toward a distal end face. That is, the cross section of the instrument head preferably decreases continuously in the distal direction until a distal end face is reached. Correspondingly, the instrument head has its smallest cross section on its distal end face, which cross section expands, preferably without jumps, to a larger cross section in the proximal direction, whereupon this largest cross section of the instrument head advantageously substantially corresponds to the cross section of the hollow shaft. An advantage of the tapering instrument head can be considered that of being able to guide the endoscopic instrument according to the invention over comparatively narrow body channels or artificially produced channels to its area of use in the body interior, since the tapering distal end of the instrument head acts like a dilator, which expands the access channel in a substantially atraumatic manner, and so the endoscopic instrument can be guided to the operation object with comparatively little resistance.

A further advantage of the distally tapering instrument head is exhibited if the instrument channels advantageously open out on the circumferential side on the tapering section of the instrument head. In this case, a fanning-out of the auxiliary instruments guided through the instrument channels can already be brought about within the inner width of the shaft or the instrument head, and so even less space is required outside of the instrument head for the fanning-out of the auxiliary instruments.

The instrument head preferably has a base body, on the distal end face of which an open mounting space is formed for holding an instrument module. The open mounting space typically extends in the proximal direction from the end face of the base body. Using an instrument module in the open mounting space of the instrument head affords the possibility of producing, in a simple fashion, instruments that are fitted to different usage functions. Different configuration variants of the instrument module can be provided for these different functions, in which configuration variants an imaging arrangement, an illumination arrangement, a force sensor, a rinsing arrangement, a suctioning arrangement, or the like can advantageously be integrated in different combinations or even on their own. The instrument module can be attached in a simple manner in the open mounting space of the base body, for example by welding or soldering. However, the instrument module is preferably adhesively bonded in the installation space.

It is particularly advantageous for the base body of the instrument head to have a monolithic design. That is, the base body consists of one piece. Methods generally referred to as “rapid prototyping” or “rapid manufacturing” can advantageously be used for producing the monolithic base body. The base body can optionally be produced from a medically approved metal, such as stainless steel or a cobalt-chromium alloy, or from plastic. In order to be able to implement the relatively complex design of the base body resulting from the instrument channels angled in the base body, metallic base bodies are preferably built layer-by-layer by a laser sintering method or by methods in which the base body material is melted on layer-by-layer using a laser. Base bodies made of plastic material are preferably produced by stereolithographic methods. All the aforementioned methods can obtain a transition-free quality of the instrument channels. Accordingly, no inaccessible corners or rear-engagements are produced in the instrument channels, in which deposits or dirt could otherwise collect, and this is particularly advantageous in respect of the hygiene required in the medical sector. Moreover, producing the base body using the aforementioned production methods in general is free of post-processing and is brought about without additional complicated assembly work.

The exit angle of the instrument channels on the instrument head preferably lies in a range between 5° and 60° with respect to the central axis of the instrument head. This angular range allows the desired fanning-out of the auxiliary instruments outside of the instrument head, with the ultimately selected exit angle of the instrument channels depending on the extent to which the shafts of the auxiliary instruments can be angled in the region of their distal end. On the instrument head, the instrument channels can all have the same exit angle or else can also have different exit angles.

According to a further advantageous embodiment of the invention, the instrument channels open out spaced apart equidistantly on the instrument head. Accordingly, the openings of adjacent instrument channels are in each case arranged offset by a same angular section with respect to the central axis of the instrument head. This arrangement allows the greatest possible distance between the instrument channels, and so the instruments guided in adjacent instrument channels cannot hinder one another, wherein the distance between the openings of adjacent instrument channels, however, in principle depends on the number of the provided instrument channels.

### BRIEF DESCRIPTION OF THE SEVERAL VIEWS OF THE DRAWINGS

The foregoing summary, as well as the following detailed description of the invention, will be better understood when read in conjunction with the appended drawings. For the purpose of illustrating the invention, there are shown in the drawings embodiments which are presently preferred. It should be understood, however, that the invention is not limited to the precise arrangements and instrumentalities shown. In the drawings:

FIG. 1 is a much simplified perspective illustration of an endoscopic instrument according to one embodiment of the invention;

FIG. 2 is a much simplified perspective illustration of an endoscopic instrument according to a second embodiment of the invention;

FIG. 3 is an enlarged side view of a base body of an instrument head of an endoscopic instrument according to FIGS. 1 and 2;

FIG. 4 is a perspective view of the base body according to FIG. 3;

FIG. 5 is a perspective view of an instrument head of an endoscopic instrument according to FIGS. 1 and 2;

FIG. 6 is a longitudinal section of the base body according to FIG. 3;

FIG. 7 is a distal end section of a separate view of instrument channels formed in the instrument head according to FIG. 5;

FIG. 8 is a separate view of the instrument channels formed in the instrument head according to FIG. 5; and

FIG. 9 is a perspective illustration of the instrument head according to FIG. 5 with auxiliary instruments emerging from the openings of the instrument channels.

### DETAILED DESCRIPTION OF THE INVENTION

The endoscopic instrument illustrated in FIG. 1 is a hollow shaft instrument having a shaft 2 formed by a rigid tube. The shaft 2 is used to guide auxiliary instruments 4. These auxiliary instruments 4 are guided through the shaft 2 from an operating part 6 arranged on the proximal end of the shaft 2, to which operating part the auxiliary instruments are coupled with actuation levers 8, to an instrument head 10 arranged on the distal end of the shaft 2.

Using the actuation levers 8, the auxiliary instruments 4 can be moved in the endoscopic instrument such that they are arranged completely within the endoscopic instrument, which is usually required when the endoscopic instrument is guided to an operating space via a body channel or an artificially produced channel, or the auxiliary instruments 4 can be moved such that distal end sections of the auxiliary instruments 4, as illustrated in FIG. 1, protrude out of the instrument head 10 of the endoscopic instrument, which is generally required in the operating space.

On the instrument head 10, the auxiliary instruments 4 are guided out of the instrument head 10, obliquely to a central axis A of instrument head 10 and shaft 2, which will be explained in more detail below. Therefore, the auxiliary instruments 4, which are likewise shaft instruments, are designed such that they can be angled obliquely with respect to the central axis of their shafts at least in one distal end section in at least one plane. The auxiliary instruments 4 can be angled such that their ends can again be brought together on the operation object like a three-finger gripper.

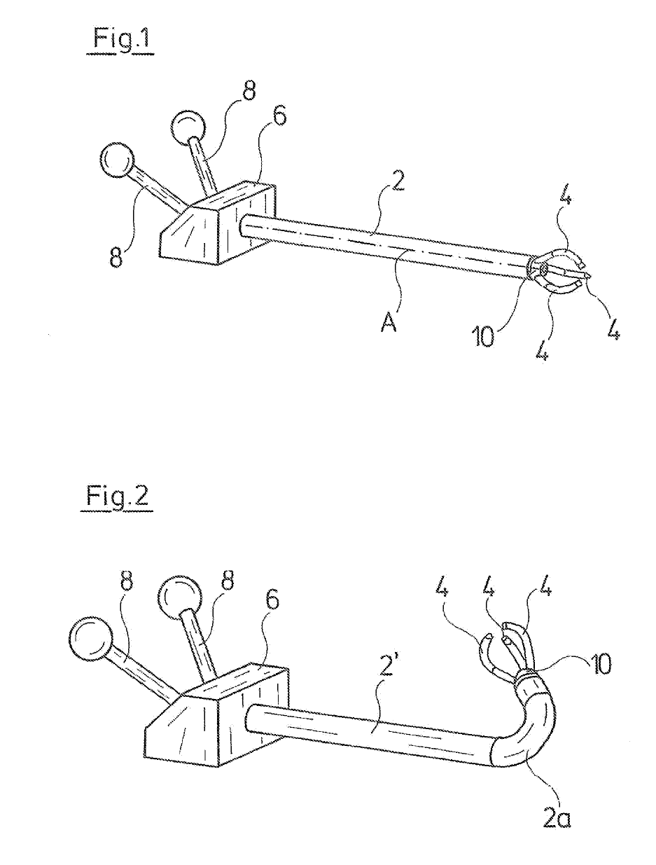

The endoscopic instrument illustrated in FIG. 2 differs from the instrument illustrated in FIG. 1 merely in that the former has a shaft 2′, in which a distal end section 2a does not have a rigid but rather a flexible design. This affords the possibility of angling or bending the end section 2a of the shaft 2′ obliquely with respect to the central axis of the remaining shaft 2′. Otherwise the instrument shown in FIG. 2 corresponds to the instrument illustrated in FIG. 1.

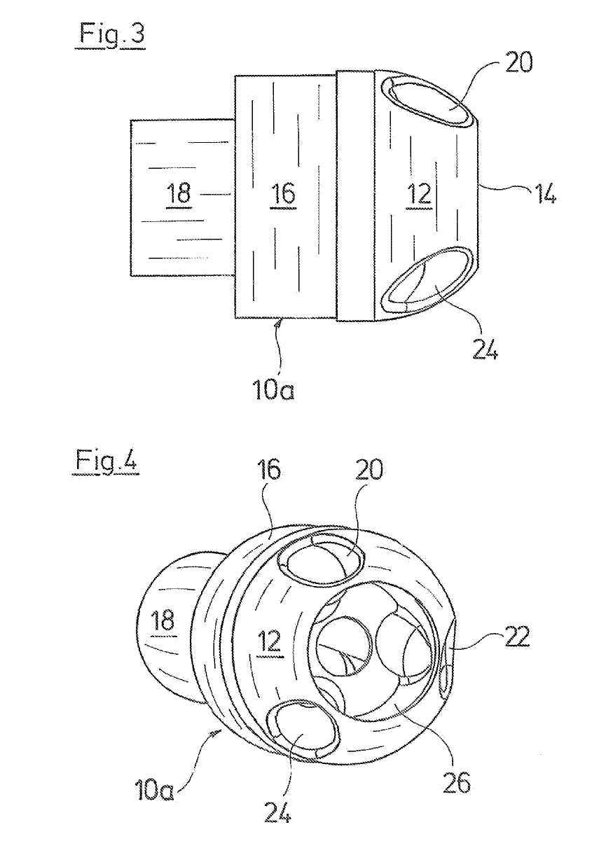

FIGS. 3, 4 and 6 illustrate a base body 10a of an instrument head 10, which is preferably provided for use in the endoscopic instruments illustrated in FIGS. 1 and 2, but can also be used in any other endoscopic instruments. This base body 10a has a monolithic design and three sections differing in terms of geometry. On the distal end, the base body 10a has a spherically arched design in a first section 12, with a spherical cap cut off to form a distal end face 14. Accordingly, in the section 12, the external cross section of the instrument head 10 decreases continuously in the distal direction to the dimensions of the end face 14. A cylindrical section 16 adjoins this section 12 on the proximal side. The section 16 is inserted into the open distal end of the shaft 2 or 2′ for attaching the instrument head 10 to the shaft 2 or 2′. The external diameter 16 is slightly smaller than the largest external diameter of the section 12 and corresponds to the internal diameter of the shaft 2 or 2′. On the proximal side, the section 16 transitions into a cylindrical section 18 in a step, the latter section having a significantly smaller external diameter than the section 16 and being arranged concentrically to the section 16.

Three instrument channels 20, 22 and 24 are formed in the instrument head 10 or the base body 10a thereof. The internal diameters of these instrument channels 20, 22 and 24 are equal in the illustrated exemplary embodiment, but in principle they can also differ. In the section 18 of the instrument head 10, the instrument channels 20, 22 and 24 are first aligned parallel to the central axis A of the instrument head 10, but already in the section 18 transition in an arced manner into a section that runs obliquely to the outside in a distal direction with an angle α (FIG. 6) with respect to the central axis A of the instrument head 10. In the exemplary embodiment illustrated in the drawing, the angle α is approximately 40°.

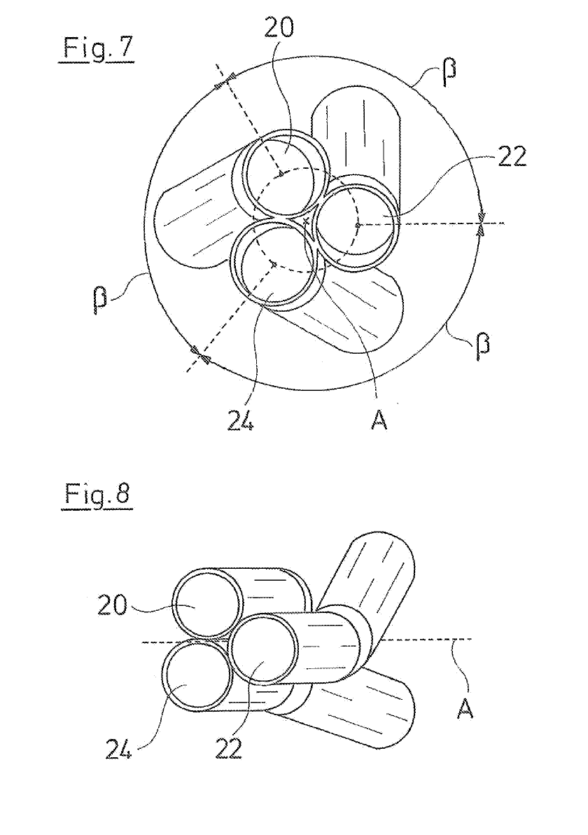

The instrument channels 20, 22 and 24 open out into the arced region on the section 12 of the base body 10a, which region adjoins the end face 14. To this extent, the instrument channels 20, 22 and 24 open out into a region that lies within the inner width of the instrument head 10 and the shaft 2 or 2′. The openings of the instrument channels 20, 22 and 24 are distributed over the circumference of the section 12, such that adjacent openings in each case have the same distance between one another. Accordingly, the central axes of adjacent instrument channels 20, 22 and 24 in each case enclose an angle β of 120° (FIG. 7) relative to the central axis A of the instrument head 10 in the illustrated exemplary embodiment.

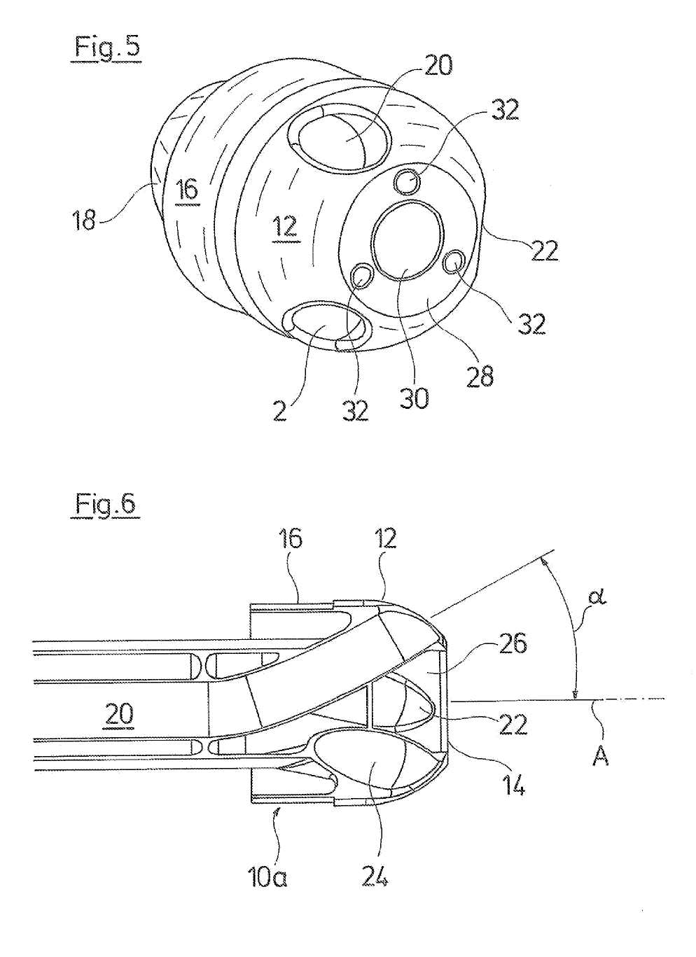

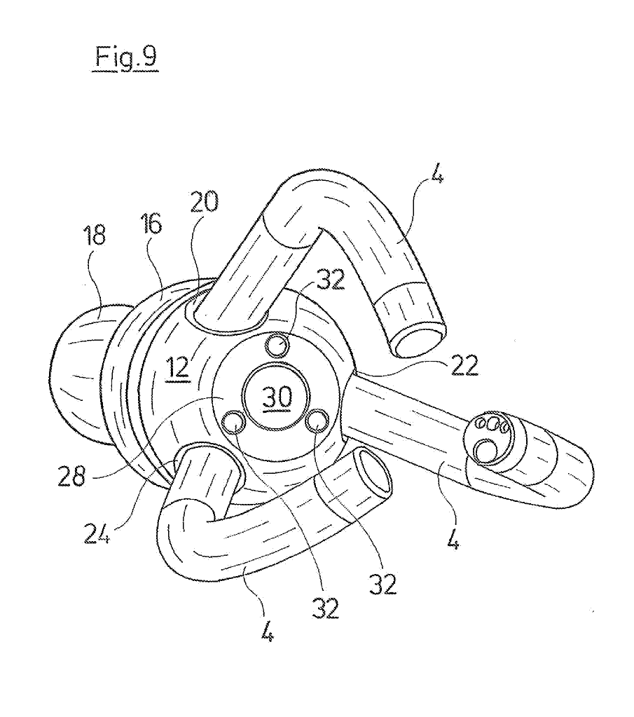

A cutout 26 is formed on the end face 14 of the distal section 12 of the base body 10a of the instrument head 10. This cutout 26 extends in a proximal direction, concentrically to the central axis A of the instrument head 10. The cutout 26 forms an installation space 26 for an instrument module 28 (FIGS. 5, 9). Integrated into the instrument module 28 there is an observation arrangement 30 and three illumination arrangements 32, as already known from the prior art. The instrument module 28 is integrally fixed in the base body 10a of the instrument head 10 by an adhesive connection such that the end face of the instrument module 28 is flush with the end face 14 of the base body 10a.

The operating space situated on the distal extension of the central axis A of the instrument head 10 is illuminated by the illumination arrangements 32 and observed using the observation arrangement 30. In the process, the arrangement of the instrument channels 20, 22 and 24 radially on the outside of the instrument module 28 and the oblique alignment of the instrument channels 20, 22 and 24 prevent the operating space from being shadowed by the auxiliary instruments 4 guided through the instrument channels 20, 22 and 24, since the auxiliary instruments 4 are first guided around the illumination and observation field on the outside or are guided in an outside edge region of the illumination and observation field and are only brought together again in the direct vicinity of the operation object.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

1.-10. (canceled)

11. An endoscopic instrument comprising a proximally arranged operating part (6), a distally arranged instrument head (10), a shaft (2, 2′) connecting the operating part (6) to the instrument head (10), and a plurality of instrument channels (20, 22, 24) guided through the shaft (2, 2′) and opening out on the instrument head (10), wherein the instrument channels (20, 22, 24) run obliquely outwardly in a distal direction with respect to a central axis (A) of the instrument head (10) in a section of the instrument channels that adjoins distal openings the instrument channels.

12. The endoscopic instrument according to claim 11, wherein the instrument channels (20, 22, 24) in the instrument head (10) transition in an arched manner from a proximal-side section aligned parallel to the central axis (A) of the instrument head (10) to a distal-side section aligned obliquely with respect to the central axis (A) of the instrument head (10).

13. The endoscopic instrument according to claim 11, wherein the instrument head (10) tapers inwardly toward a distal end face (14).

14. The endoscopic instrument according to claim 13, wherein the instrument channels (20, 22, 24) open out on a circumferential side of a tapering section (12) of the instrument head (10).

15. The endoscopic instrument according to claim 11, wherein the instrument head (10) has a base body (10a), and wherein a distal end face (14) of the base body has an open installation space (26) for holding an instrument module (28).

16. The endoscopic instrument according to claim 15, wherein the instrument module (28) is adhesively bonded in the installation space (26).

17. The endoscopic instrument according to claim 15, wherein an observation arrangement (30) and/or illumination arrangement (32) is integrated into the instrument module (28).

18. The endoscopic instrument according to claim 15, wherein the base body (10a) of the instrument head (10) has a monolithic design.

19. The endoscopic instrument according to claim 11, wherein an exit angle (α) of the instrument channels (20, 22, 24) on the instrument head (10) lies in a range between 5° and 60° relative to the central axis (A) of the instrument head (10).

20. The endoscopic instrument according to claim 11, wherein the instrument channels (20, 22, 24) open out spaced apart equidistantly on a circumference of the instrument head (10).
